(19) United States
(12) Patent Application Publication
(10) Pub. No.: US 2016/0022227 A1
(43) Pub. Date: Jan. 28, 2016

(54) Method of Transmitting an Emergency Audiovisual Alert to an Emergency Contact and Emergency Medical Services

(71) Applicant: Andrew Chen, Palm Coast, FL (US)

(72) Inventor: Andrew Chen, Palm Coast, FL (US)

(21) Appl. No.: 14/803,458

(22) Filed: Jul. 20, 2015

Related U.S. Application Data
(60) Provisional application No. 62/027,477, filed on Jul. 22, 2014.

(51) Int. Cl. A61B 5/00; G08B 25/01; A61B 5/01; A61B 5/08; A61B 5/024; A61B 5/021

(52) U.S. Cl. CPC A61B 5/747; A61B 5/024; A61B 5/021; A61B 5/01; A61B 5/0816; A61B 5/7282; G08B 25/016

(57) ABSTRACT

A method of transmitting an emergency audiovisual alert to an emergency contact and emergency medical services allows a user who is experiencing a medical emergency to notify an emergency contact and/or emergency medical services. An emergency alert process is initiated via a plurality of failsafe triggers. Audiovisual data and location data from a user computing device are sent to an at least one recipient device of the emergency contact and emergency medical services. This allows emergency medical services to provide a more educated and potentially life-saving response. Audiovisual data and location data are captured through an audio-capture device, a video-capture device, and a geospatial positioning module. The plurality of failsafe triggers includes a haptic distress input, a vocal distress command, and a vital sign reading. As such, the emergency alert process may be initiated even if the user is unconscious or otherwise incapacitated.

The current application claims a priority to the U.S. Provisional Patent application Ser. No. 62/027,477 filed on Jul. 22, 2014.

### FIELD OF THE INVENTION

The present invention relates generally to a method of improving the response to a medical emergency. More specifically, the present invention is a method of transmitting an emergency alert, both video as well as audio, to emergency medical services in addition to one or more emergency contacts.

### BACKGROUND OF THE INVENTION

The advancement of medical technology has greatly improved the ability to treat diseases and save lives as well as improved the quality of life in general. Emergency service infrastructure has improved as well as the ability to quickly respond to all manner of medical emergencies. Accessibility to emergency services has improved as well due to the prevalence of computing devices such as smartphones. Despite these massive improvements in medical services and accessibility, certain groups of people are still at risk of experiencing a medical emergency and being unable to call for assistance. For example, a person who falls, becomes unconscious, unable to move, or similarly incapacitated is at significant risk. Certain medical emergencies may affect a person's ability to speak and articulate his or her predicament to emergency services. Conventional medical emergency alert devices generally require some form of input from the user in order to activate and do not account for the possibility of the user being unable to contact emergency services on his or her own.

The present invention is a method of transmitting an emergency audiovisual alert to an emergency contact and emergency medical services. The audiovisual alert is transmitted from a user computing device to an at least one recipient computing device and may be activated manually. However, the audiovisual alert may also be activated automatically in the event of incapacitation such that the audiovisual alert cannot be activated manually. The present invention is primarily intended to provide emergency medical services with a real-time audiovisual stream that provides an overview of an emergency situation. In addition to emergency medical services, the audiovisual alert may be transmitted to one or more emergency contacts as well. The audiovisual alert additionally includes the location of the user computing device. The audiovisual and location data is captured utilizing hardware that is native to the user computing device. The in-depth knowledge of the emergency situation provided by the present invention allows emergency medical services to obtain a better assessment of the emergency situation prior to the arrival of first responders on an emergency scene, and provide a more educated response as a result. This is critical due to the fact that time is a crucial factor when responding to a real life emergency. The information provided to emergency medical services by the present invention greatly improves the efficiency of emergency medical services as well as the ability to save lives.

### BRIEF DESCRIPTION OF THE DRAWINGS

FIG. 1 is a diagrammatic overview of the present invention.

FIG. 2A is a flowchart illustrating the overall process that is followed by the present invention.

FIG. 2B is a continuation of the flowchart illustrated in FIG. 2A.

FIG. 3 is a diagram of an example haptic distress input on the user computing device.

FIG. 4 is a flowchart of the secondary process that is followed by the present invention when utilizing a vocal distress command to initiate the emergency alert process.

FIG. 5 is a diagram of an example recording process for a vocal distress command.

FIG. 6 is a flowchart of the secondary process that is followed by the present invention when utilizing a vital sign monitoring device and a life-threatening vital sign maximum threshold to initiate the emergency alert process.

FIG. 7 is a flowchart of the secondary process that is followed by the present invention when utilizing a vital sign monitoring device and a life-threatening vital sign minimum threshold to initiate the emergency alert process.

FIG. 8 is a diagram of an example established life-threatening vital sign maximum threshold and an example established life-threatening vital sign minimum threshold.

FIG. 9 is a flowchart of the secondary process that is followed by the present invention when inputting emergency contact information.

FIG. 10 is a flowchart of the secondary process that is followed by the present invention when providing user personal information.

### DETAIL DESCRIPTIONS OF THE INVENTION

All illustrations of the drawings are for the purpose of describing selected versions of the present invention and are not intended to limit the scope of the present invention.

The present invention is a method of transmitting an emergency audiovisual alert to an emergency contact and emergency medical services. The present invention allows a user to notify an emergency contact as well as emergency medical services when he or she is experiencing a medical emergency. The emergency contact and emergency medical services are provided with a live audiovisual stream of the user's medical predicament, allowing for a more calculated and appropriate response to the situation.

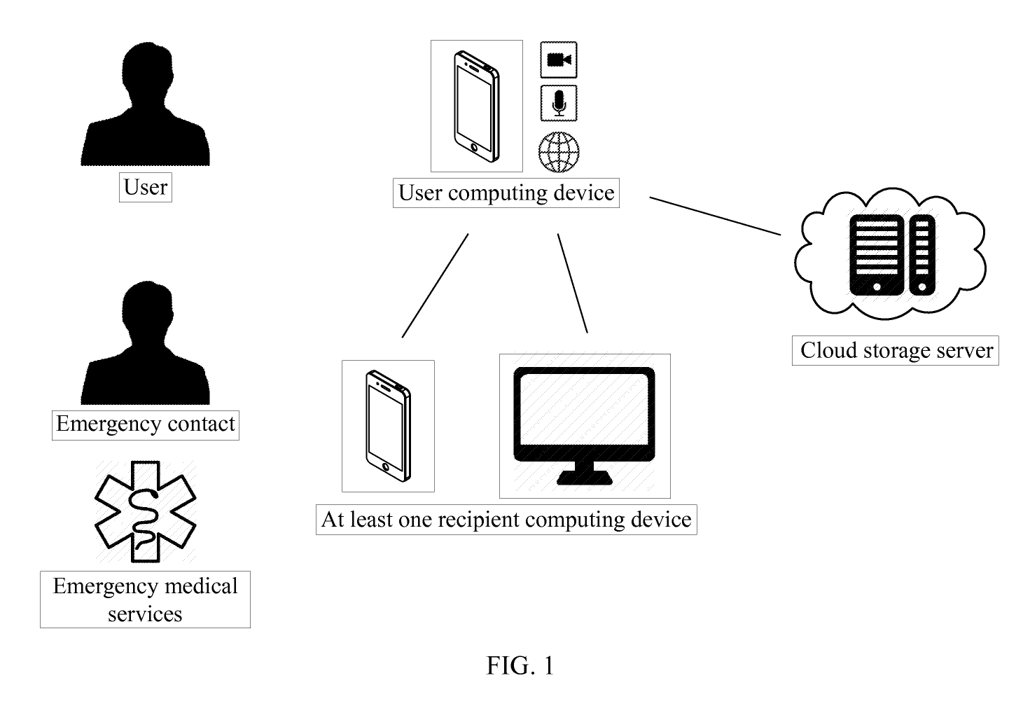

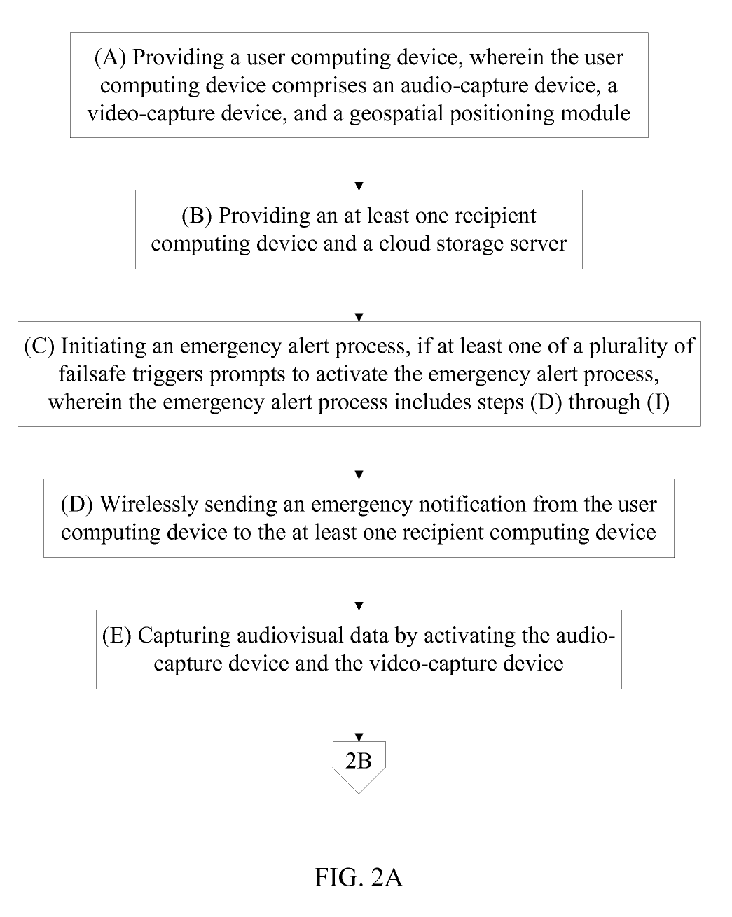

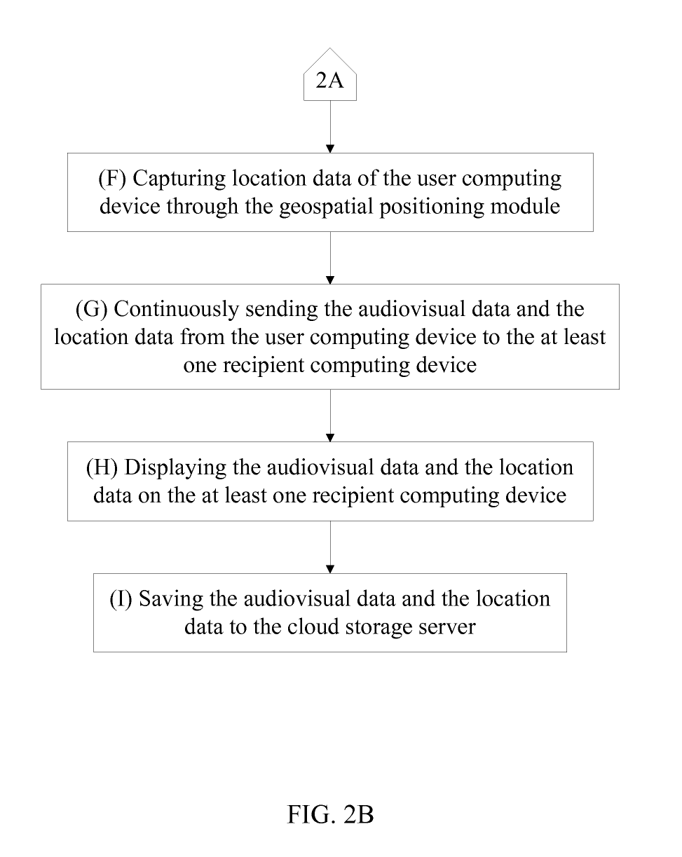

A diagrammatic overview of the present invention is shown in FIG. 1 while the overall process that is followed by the present invention is shown in FIG. 2A and FIG. 2B. The present invention utilizes a user computing device such as, but not limited to a smartphone. The present invention utilizes hardware that is native to the user computing device including an audio-capture device, a video-capture device, and a geospatial positioning module. In the context of a smartphone, the audio-capture device is a microphone that is capable of capturing audio in the user's vicinity. The video-capture device is a camera that is capable of capturing video of the user's surroundings. Finally, the geospatial positioning module is a Global Positioning System (GPS) module that is capable of determining the geographical location of the user computing device. The present invention additionally utilizes an at least one recipient computing device as well as a cloud storage server. The at least one recipient computing device receives audiovisual data and location data from the user computing device. The at least one recipient computing device may be a smartphone, an emergency medical services dispatcher computer, or a similar device.

When the user experiences a medical emergency, an emergency alert process is initiated. A plurality of failsafe triggers prompts to activate the emergency alert process. The emergency alert process is initiated if at least one of the plurality of failsafe triggers prompts to activate the emergency alert process. The plurality of failsafe triggers is designed to ensure that the user is able to notify the emergency contact and emergency medical services that he or she is experiencing a medical emergency regardless of whether or not the user is able to physically initiate the emergency alert process. After the emergency alert process is initiated, an emergency notification is wirelessly sent from the user computing device to the at least one recipient computing device. The emergency notification notifies the emergency contact and emergency medical services that the user is experiencing a medical emergency. The emergency notification may be sent via multiple channels of communication including, but not limited to, Short Message Service (SMS), email, and telephone call. Audiovisual data from the user's vicinity is captured by activating the audio-capture device and the video-capture device. Additionally, location data of the user computing device is captured through the geospatial positioning module. The audiovisual data and the location data are continuously sent from the user computing device to the at least one recipient computing device. The audiovisual data and the location data are displayed on the at least one recipient computing device. The emergency contact and emergency medical services are thus provided with a live audiovisual stream of the user's predicament, allowing for a more calculated and appropriate response. The location data allows the emergency contact and emergency medical services to quickly respond to the user's location as well. The audiovisual data and the location data are saved to the cloud storage server. This allows the user, the emergency contact, and emergency medical services to access the saved audiovisual data and the location data as required. Additionally, the cloud storage server prevents data loss in the event that the user computing device and/or the at least one recipient computing device become damaged or otherwise compromised.

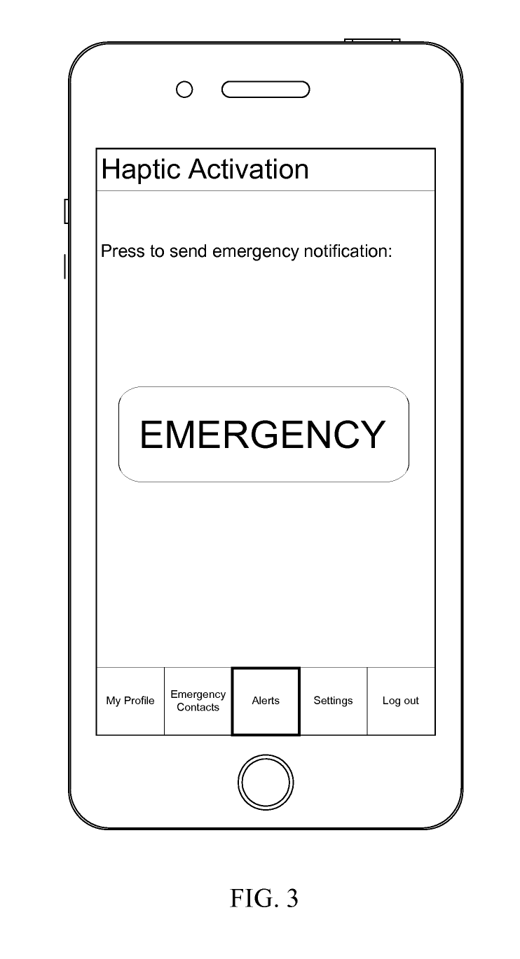

With reference to FIG. 3, a haptic distress input may be received through the user computing device. The haptic distress input is one of the plurality of failsafe triggers and is utilized by the user if the user is physically able to initiate the emergency alert process. The haptic distress input may be a one-touch switch that is activated on a touchscreen of the user computing device, a short alphanumeric code input on the user computing device, or a similar input. Once the haptic distress input is received, the emergency alert process is initiated.

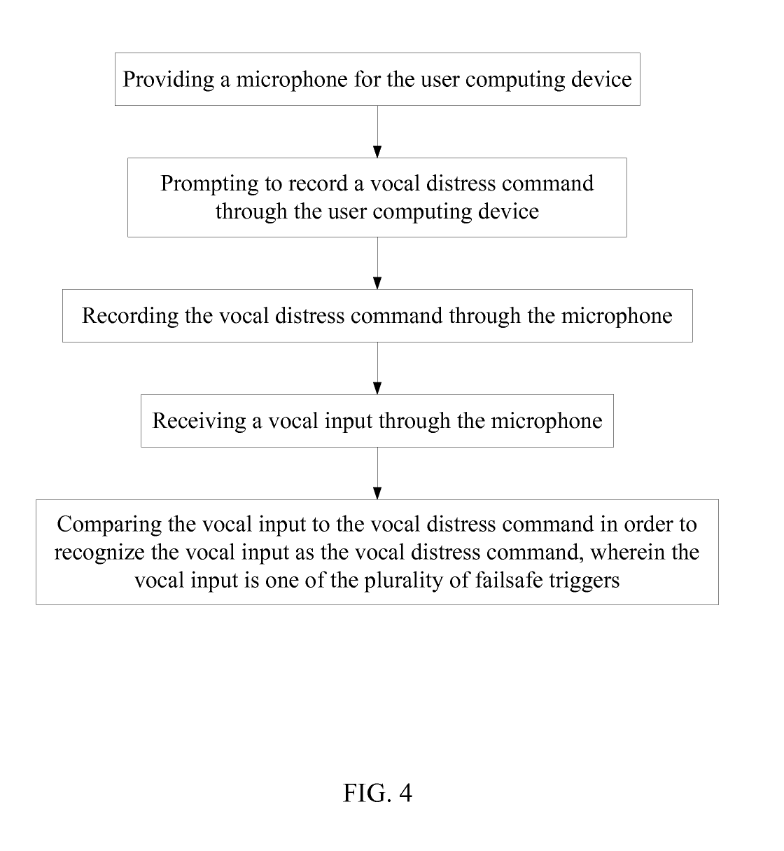

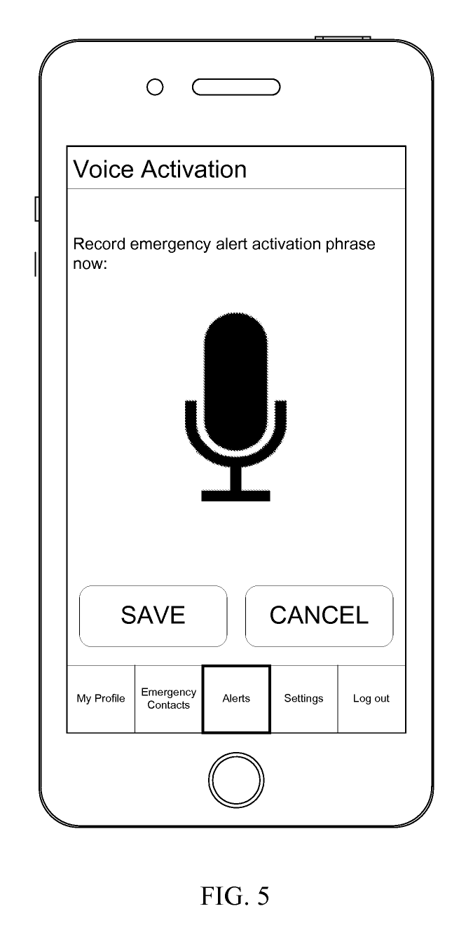

If the user is unable to physically initiate the emergency alert process, a vocal distress command may be utilized to initiate the emergency alert process. With reference to FIG. 4 and FIG. 5, in the preferred embodiment of the present invention, a microphone for the user computing device is utilized in order to both record and capture the vocal distress command. When initially setting up the vocal distress command, the user is prompted to record the vocal distress command through the user computing device and the vocal distress command is recorded through the microphone. After the vocal distress command has been recorded and a vocal input is received through the microphone, the vocal input is compared to the vocal distress command. This allows the vocal input to be recognized as the vocal distress command. The vocal input is one of the plurality of failsafe triggers and is able to initiate the emergency alert process. This allows the user to initiate the emergency alert process even if he or she is physically unable to do so. For example, if the user is immobilized and unable to reach the user computing device to provide the haptic distress input, the vocal input may be utilized instead to initiate the emergency alert process.

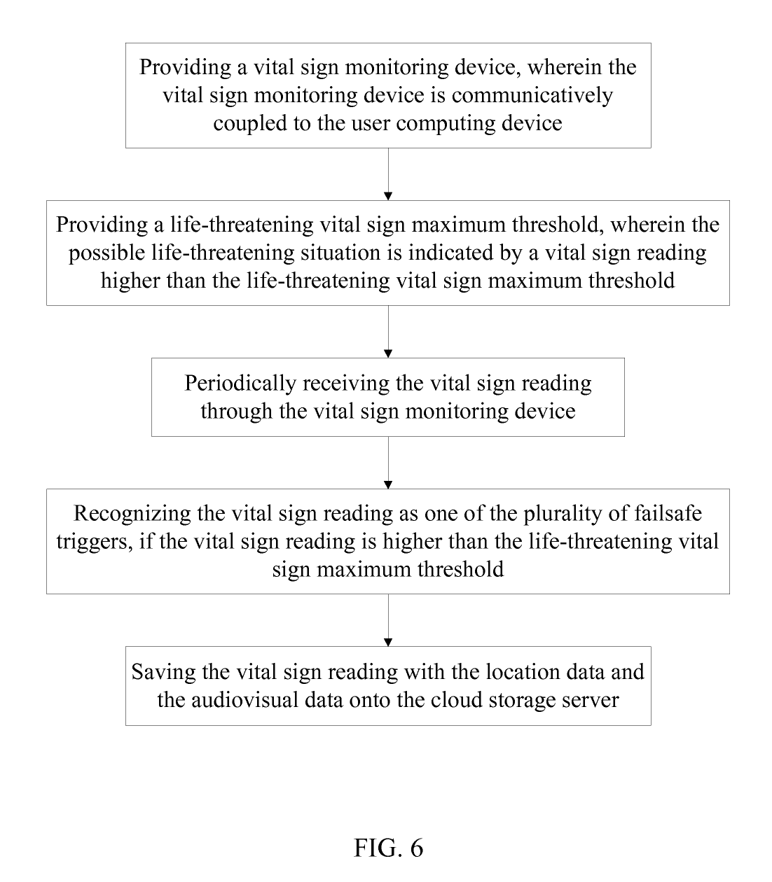

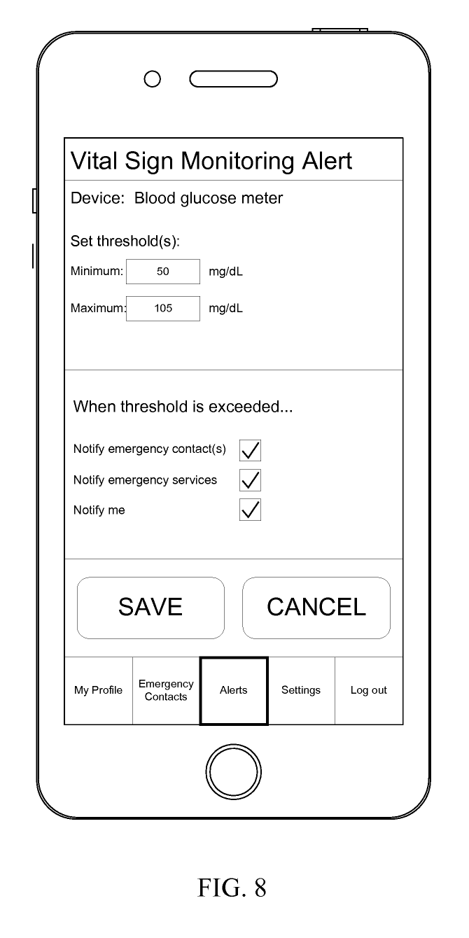

Although the vocal input provides a means of initiating the emergency alert process if the user is unable to physically provide the haptic distress input, the vocal input does not account for the fact that the user may become unconscious and unable to provide the vocal input as well. As shown in FIGS. 6-8, the present invention utilizes a vital sign monitoring device that is communicatively coupled to the user computing device. Wireless technology such as, but not limited to, Bluetooth, may be utilized to communicatively couple the vital sign monitoring device to the user computing device. The most common type of vital sign monitoring device is a blood glucose meter, although additional types of vital sign monitoring devices may be utilized with the present invention as well. A life-threatening vital sign maximum threshold is established such that the vital sign monitoring device is able to determine if the user is in a possible life-threatening situation. With respect to FIG. 6, the possible life-threatening situation is indicated by a vital sign reading higher than the life-threatening vital sign maximum threshold. An example of this is if the user's blood glucose level becomes too high.

Because the vital sign reading is subject to fluctuation over time, the vital sign reading is periodically received through the vital sign monitoring device. It is possible for the user to fall unconscious or become similarly incapacitated due to a vital sign reading that is higher than the life-threatening vital sign maximum threshold. As such, the vital sign reading is recognized as one of the plurality of failsafe triggers if the vital sign reading is higher than the life-threatening vital sign maximum threshold. This enables the emergency alert process to be initiated even if the user has fallen unconscious or is similarly incapacitated. The vital sign reading is saved with the location data and the audiovisual data onto the cloud storage server. As such, the user, the emergency contact, and emergency medical services may access the user's historical vital sign readings as needed. The vital sign reading data may be organized and presented in a manner such that the user's medical history with relation to the vital sign reading is easily visible.

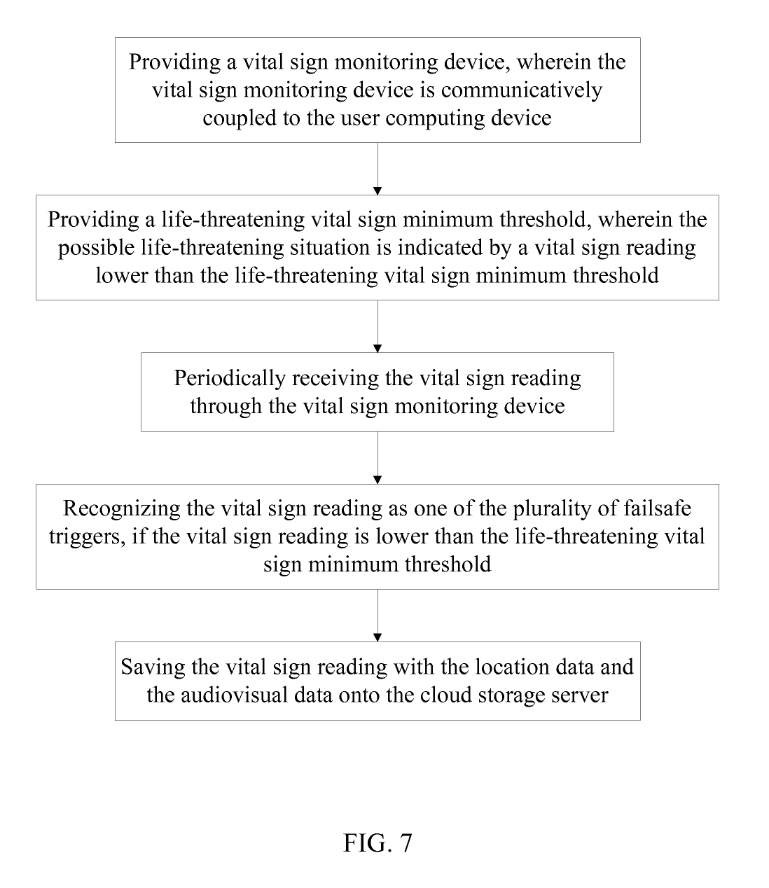

With respect to FIG. 7, a life-threatening minimum threshold is established such that the vital sign monitoring device is able to determine if the user is in a possible life-threatening situation. The possible life-threatening situation is indicated by a vital sign reading lower than the life-threatening vital sign minimum threshold. For example, the user's blood glucose level may become too low. The vital sign reading is periodically received through the vital sign monitoring device to allow for continuous monitoring of the user's vital sign reading. The vital sign reading is recognized as one of the plurality of failsafe triggers if the vital sign reading is lower than the life-threatening vital sign minimum threshold. As before, this enables the emergency alert process to be initiated even if the user has fallen unconscious or is similarly incapacitated. The vital sign reading is transmitted and saved with the location data and the audiovisual data onto the cloud storage server for future access as needed and to prevent loss of the vital sign reading data.

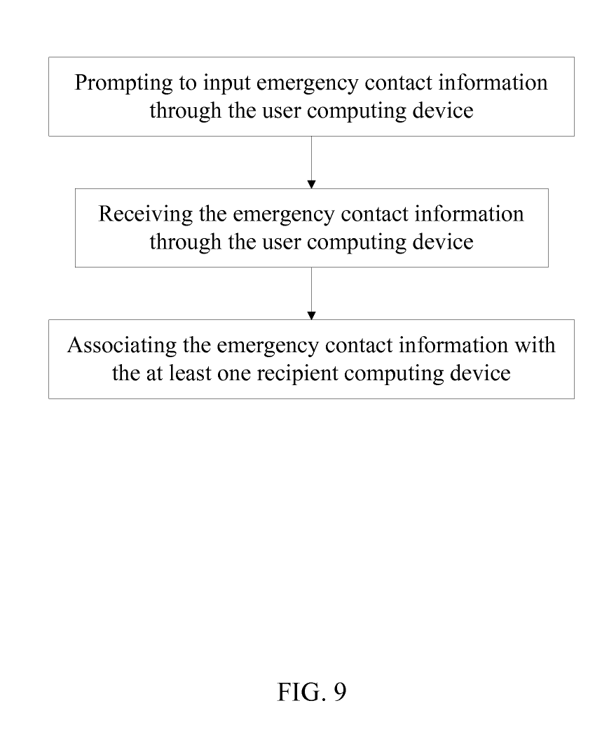

The user may designate multiple emergency contacts to which the emergency notification is sent. As shown in FIG. 9, the user is prompted to input emergency contact information through the user computing device and the emergency contact is notified if the emergency alert process is initiated. The emergency contact information is received through the user computing device and is associated with the at least one recipient computing device. This enables the at least one recipient computing device to receive the audiovisual stream, the location data, and the vital sign reading (if applicable) from the user computing device when the emergency alert process is initiated.

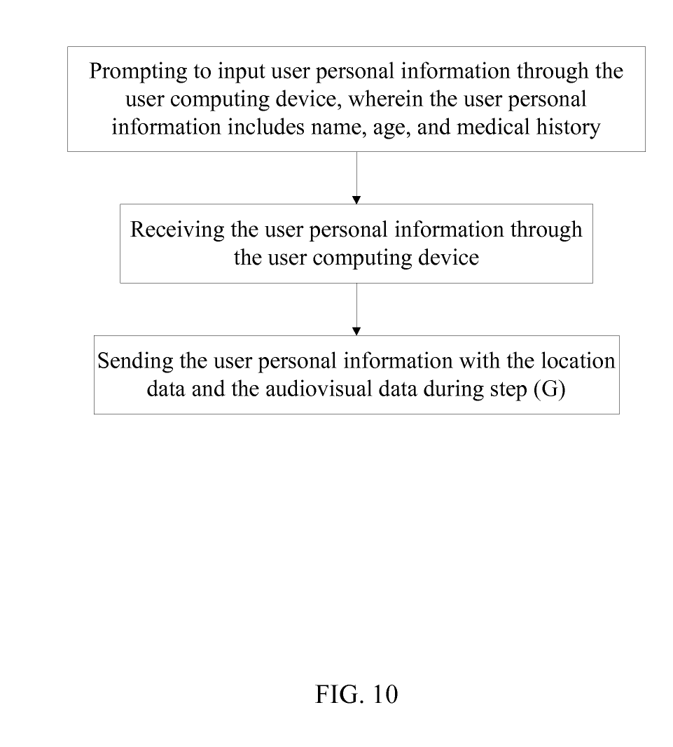

In order to further improve the ability for medical emergency services to more accurately assess the user's medical emergency, the user is able to provide his or her personal information. As shown in FIG. 10, the user is prompted to input user personal information through the user computing device. The user personal information includes name, age, and medical history. Medical emergency services is thus able to take this information into account when assessing the user's medical emergency and calculating the most appropriate response to the emergency. The user personal information is received through the user computing device and is sent to the at least one recipient computing device with the location data and the audiovisual data. The user personal information increases the ability for medical emergency services to provide a more educated and potentially life-saving response to the user's medical emergency.

Although the present invention has been explained in relation to its preferred embodiment, it is understood that many other possible modifications and variations can be made without departing from the spirit and scope of the present invention as hereinafter claimed.

What is claimed is:

1. A method of transmitting an emergency audiovisual alert to an emergency contact and emergency medical services, the method comprises the steps of:
(A) providing a user computing device, wherein the user computing device comprises an audio-capture device, a video-capture device, and a geospatial positioning module;
(B) providing an at least one recipient computing device and a cloud storage server;
(C) initiating an emergency alert process, if at least one of a plurality of failsafe triggers prompts to activate the emergency alert process, wherein the emergency alert process includes steps (D) through (I);
(D) wirelessly sending an emergency notification from the user computing device to the at least one recipient computing device;
(E) capturing audiovisual data by activating the audio-capture device and the video-capture device;
(F) capturing location data of the user computing device through the geospatial positioning module;
(G) continuously sending the audiovisual data and the location data from the user computing device to the at least one recipient computing device;
(H) displaying the audiovisual data and the location data on the at least one recipient computing device; and
(I) saving the audiovisual data and the location data to the cloud storage server.

2. The method of transmitting an emergency audiovisual alert to an emergency contact and emergency medical services, the method as claimed in claim 1 comprises the steps of:
receiving a haptic distress input through the user computing device, wherein the haptic distress input is one of the plurality of failsafe triggers.

3. The method of transmitting an emergency audiovisual alert to an emergency contact and emergency medical services, the method as claimed in claim 1 comprises the steps of:
providing a microphone for the user computing device;
prompting to record a vocal distress command through the user computing device;
recording the vocal distress command through the microphone;
receiving a vocal input through the microphone; and
comparing the vocal input to the vocal distress command in order to recognize the vocal input as the vocal distress command, wherein the vocal input is one of the plurality of failsafe triggers.

4. The method of transmitting an emergency audiovisual alert to an emergency contact and emergency medical services, the method as claimed in claim 1 comprises the steps of:
providing a vital sign monitoring device, wherein the vital sign monitoring device is communicatively coupled to the user computing device;
providing a life-threatening vital sign maximum threshold, wherein a possible life-threatening situation is indicated by a vital sign reading higher than the life-threatening vital sign maximum threshold;
periodically receiving the vital sign reading through the vital sign monitoring device;
recognizing the vital sign reading as one of the plurality of failsafe triggers,
if the vital sign reading is higher than the life-threatening vital sign maximum threshold; and
saving the vital sign reading with the location data and the audiovisual data onto the cloud storage server.

5. The method of transmitting an emergency audiovisual alert to an emergency contact and emergency medical services, the method as claimed in claim 1 comprises the steps of:
providing a vital sign monitoring device, wherein the vital sign monitoring device is communicatively coupled to the user computing device;
providing a life-threatening vital sign minimum threshold, wherein a possible life-threatening situation is indicated by a vital sign reading lower than the life-threatening vital sign minimum threshold;
periodically receiving the vital sign reading through the vital sign monitoring device;
recognizing the vital sign reading as one of the plurality of failsafe triggers,
if the vital sign reading is lower than the life-threatening vital sign minimum threshold; and
saving the vital sign reading with the location data and the audiovisual data onto the cloud storage server.

6. The method of transmitting an emergency audiovisual alert to an emergency contact and emergency medical services, the method as claimed in claim 1 comprises the steps of:
prompting to input emergency contact information through the user computing device;
receiving the emergency contact information through the user computing device; and
associating the emergency contact information with the at least one recipient computing device.

7. The method of transmitting an emergency audiovisual alert to an emergency contact and emergency medical services, the method as claimed in claim 1 comprises the steps of:
prompting to input user personal information through the user computing device, wherein the user personal information includes name, age, and medical history;
receiving the user personal information through the user computing device; and
sending the user personal information with the location data and the audiovisual data during step (G).
